(19) United States
(12) Patent Application Publication
(10) Pub. No.: US 2025/0375204 A1
(43) Pub. Date: Dec. 11, 2025

(54) ATRAUMATIC IMPLANTATION AND CLOSURE OF RECONSTRUCTIVE LAAC DEVICE FOR SAFETY AND EFFICACY

(71) Applicant: BOSTON SCIENTIFIC SCIMED, INC., MAPLE GROVE, MN (US)

(72) Inventors: Jerry Sy-Shyan Wang, Edina, MN (US); Philip Andrew Litecky, Forest Lake, MN (US); Joshua David Tillman, New Brighton, MN (US); Adam Troy Olson, Plymouth, MN (US)

(73) Assignee: BOSTON SCIENTIFIC SCIMED, INC., MAPLE GROVE, MN (US)

(21) Appl. No.: 19/227,801

(22) Filed: Jun. 4, 2025

Related U.S. Application Data
(60) Provisional application No. 63/656,227, filed on Jun. 5, 2024.

(51) Int. Cl. A61B 17/12

(52) U.S. Cl. CPC A61B 17/12031; A61B 17/12122; A61B 17/12172; A61B 2017/1205

(57) ABSTRACT

An implant for left atrial appendage closure includes an expandable framework with a collar, a shaft, a rod, and a lock. The expandable framework is configured to expand from a collapsed delivery configuration to an expanded deployed configuration. The shaft is disposed within the collar which is axially moveable relative to the shaft. The rod is disposed within the shaft and is coupled to the collar. The lock is coupled to the shaft and includes an engagement member configured to move between a proximally facing constrained configuration and a distally facing radially expanded configuration. The rod is coupled to the collar such that rotation of the rod pulls the collar and expandable framework proximally over the shaft toward the lock, and the expandable framework is configured to rotate and move axially independently of the lock.

### CROSS-REFERENCE TO RELATED APPLICATION

This application is a continuation of U.S. Patent Application Ser. No. 63/656,227, filed Jun. 5, 2024, entitled “ATRAUMATIC IMPLANTATION AND CLOSURE OF RECONSTRUCTIVE LAAC DEVICE FOR SAFETY AND EFFICACY”, which is incorporated by reference herein in its entirety.

### TECHNICAL FIELD

The disclosure pertains to medical devices and more particularly to devices for left atrial appendage occlusion, and methods for using such medical devices.

### BACKGROUND

A wide variety of medical devices have been developed for medical use including, for example, medical devices utilized to occlude regions of the body. These medical devices may be used in a variety of body regions including the left atrial appendage (LAA). In patients suffering from atrial fibrillation, the LAA may not properly contract or empty, causing stagnant blood to pool within its interior, which can lead to the undesirable formation of thrombi within the LAA.

Thrombi forming in the LAA may break loose from this area and enter the blood stream. Thrombi that migrate through the blood vessels may eventually plug a smaller vessel downstream and thereby contribute to stroke or heart attack. Clinical studies have shown that the majority of blood clots in patients with atrial fibrillation originate in the LAA. As a treatment, left atrial appendage closure (LAAC) is a procedure that blocks or closes the opening to the LAA to keep blood clots from leaving there and the bloodstream. Various medical devices have been developed which are deployed to close off the LAA. Of the known medical devices and methods, each has certain advantages and disadvantages. There is an ongoing need to provide alternative medical devices as well as alternative methods for manufacturing and using medical devices.

### SUMMARY

This disclosure provides design, material, manufacturing method, and use alternatives for medical devices. An example implant for left atrial appendage closure includes an expandable framework having a proximal end coupled to a collar, the expandable framework configured to expand from a collapsed delivery configuration to an expanded deployed configuration, a shaft disposed within the collar wherein the collar is axially moveable relative to the shaft, a rod disposed within the shaft and coupled to the collar, and a lock coupled to the shaft, the lock including an engagement member configured to move between a proximally facing constrained configuration and a distally facing radially expanded configuration. The rod is coupled to the collar such that rotation of the rod pulls the collar and expandable framework proximally over the shaft toward the lock, wherein the expandable framework is configured to rotate and move axially independently of the lock.

Alternatively or additionally to the embodiment above, the rod is threaded and the implant further comprises a nut threadingly engaged around the rod and coupled to the collar.

Alternatively or additionally to any of the embodiments above, the collar and nut each have at least one opening and the openings are aligned, the implant further including at least one pin extending through the openings in the collar and nut and having an end positioned in the threading on the rod.

Alternatively or additionally to any of the embodiments above, the engagement member is biased in the distally facing radially expanded configuration.

Alternatively or additionally to any of the embodiments above, the engagement member is fixed to the proximal end of the shaft and includes a plurality of arms.

Alternatively or additionally to any of the embodiments above, each arm has a first end fixed to the proximal end of the shaft and an opposing second end, wherein in the collapsed delivery configuration each arm extends proximally from the proximal end of the shaft, and in the expanded deployed configuration, each arm bends distally with the second end of each arm extending distally and spaced apart radially from the shaft.

Alternatively or additionally to any of the embodiments above, the plurality of arms is interconnected to define a plurality of diamond shapes.

Alternatively or additionally to any of the embodiments above, the plurality of arms forms a plurality of petals each having a base fixed to the shaft and an opposing free end, each petal defined by two arms extending between the base and the free end with an opening therebetween.

Alternatively or additionally to any of the embodiments above, each petal is independently movable relative to the shaft and adjacent petals, each petal being free of connection to any adjacent petal.

Alternatively or additionally to any of the embodiments above, after the expandable framework is moved to the expanded deployed configuration, the expandable framework is configured to be rotated and then pulled proximally over the shaft.

Alternatively or additionally to any of the embodiments above, the expandable framework includes a plurality of struts each having a proximal end, a middle portion, and distal end, wherein the proximal ends are coupled to the collar, the distal ends are coupled together, and the middle portions are moveable between the collapsed delivery configuration and the expanded deployed configuration, wherein the plurality of struts are biased in the expanded deployed configuration.

Alternatively or additionally to any of the embodiments above, at least the middle portion of each strut has a plurality of projections extending laterally from the strut.

An example implant assembly for left atrial appendage closure includes a delivery sheath defining a lumen, a rotator sheath slidably disposed within the lumen of the delivery sheath, an actuation sheath disposed within a lumen of the rotator sheath, an implant releasably coupled to a distal end of the rotator sheath and the actuation sheath, the implant including an expandable framework configured to expand from a collapsed delivery configuration to an expanded deployed configuration, a shaft slidably disposed within at least a portion of the expandable framework, a rod disposed within the shaft and coupled to the expandable framework and the actuation sheath, and a lock coupled to a proximal end of the shaft, the lock including an engagement member configured to move between a proximally facing constrained configuration and a distally facing radially expanded configuration.

Alternatively or additionally to the embodiment above, the distal end of the rotator sheath has a plurality of distally extending fingers configured to engage the engagement member.

Alternatively or additionally to any of the embodiments above, the actuation sheath is configured to be rotated to rotate the rod which pulls the expandable framework proximally over the shaft and rod.

Alternatively or additionally to any of the embodiments above, the rod is threaded and has a proximal end releasably coupled to the actuation sheath.

Alternatively or additionally to any of the embodiments above, the engagement member is fixed to the proximal end of the shaft and includes a plurality of arms.

Alternatively or additionally to any of the embodiments above, each arm has a first end fixed to the proximal end of the shaft and an opposing second end, wherein in the collapsed delivery configuration each arm extends proximally from the proximal end of the shaft, and in the expanded deployed configuration, each arm bends distally with the second end of each arm extending distally and spaced apart radially from the shaft.

Alternatively or additionally to any of the embodiments above, the plurality of arms is interconnected to define a plurality of diamonds forming a star shape.

An example method of closing a left atrial appendage includes coupling a delivery system to an implant, the implant including an expandable framework having a proximal end coupled to a collar, the expandable framework configured to expand from a collapsed delivery configuration to an expanded deployed configuration, at least a portion of the expandable framework having a plurality of projections extending laterally therefrom, a shaft disposed within the collar and axially moveable relative to the collar, a lock coupled to the shaft, the lock including an engagement member configured to move between a proximally facing constrained configuration and a distally facing radially expanded configuration, wherein the expandable framework is in the collapsed delivery configuration and the engagement member is in the proximally facing constrained configuration within the delivery system. The method further including inserting the delivery system through a patient's vasculature until the expandable framework, in the collapsed delivery configuration, is positioned inside the left atrial appendage, withdrawing the delivery system from the expandable framework to allow the expandable framework to expand to the expanded deployed configuration, rotating at least a portion of the delivery system to rotate the expandable framework and engage the plurality of projections with an inner surface of the left atrial appendage and twist a neck of the left atrial appendage around the shaft, withdrawing the delivery system from the lock to allow the engagement member to move into the distally facing radially expanded configuration adjacent a proximal face of the twisted neck of the left atrial appendage, pulling the expandable framework proximally until the twisted neck of the left atrial appendage is pinched between the expandable framework and the engagement member, and disengaging the delivery system from the implant.

The above summary of some embodiments, aspects, and/or examples is not intended to describe each embodiment or every implementation of the present disclosure. The figures and the detailed description which follows more particularly exemplify these embodiments.

### BRIEF DESCRIPTION OF THE DRAWINGS

The disclosure may be more completely understood in consideration of the following detailed description of various embodiments in connection with the accompanying drawings, in which:

FIG. 1 is a side cross-sectional view of an example implant and delivery system;

FIGS. 2A and 2B are side cross-sectional views of the implant and delivery system of FIG. 1 with the delivery sheath partially withdrawn and fully withdrawn, respectively, and the implant partially expanded;

FIGS. 3-5 are perspective views of the implant and delivery system of FIG. 1 during deployment;

FIGS. 6-11 are side cross-sectional views of the implant and delivery system of FIG. 1 during deployment;

FIG. 12 shows the engagement member of the implant positioned on the proximal face of a twisted left atrial appendage after implantation; and

FIGS. 13A and 13B show other examples of engagement members of the implant positioned on the proximal face of a twisted left atrial appendage after implantation.

While aspects of the disclosure are amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

The following description should be read with reference to the drawings, which are not necessarily to scale, wherein like reference numerals indicate like elements throughout the several views. The detailed description and drawings are intended to illustrate but not limit the disclosure. Those skilled in the art will recognize that the various elements described and/or shown may be arranged in various combinations and configurations without departing from the scope of the disclosure. The detailed description and drawings illustrate example embodiments of the disclosure.

For the following defined terms, these definitions shall be applied, unless a different definition is given in the claims or elsewhere in this specification.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5). Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For simplicity and clarity purposes, not all elements of the disclosure are necessarily shown in each figure or discussed in detail below. However, it will be understood that the following discussion may apply equally to any and/or all of the components for which there are more than one, unless explicitly stated to the contrary. Additionally, not all instances of some elements or features may be shown in each figure for clarity.

Relative terms such as “proximal”, “distal”, “advance”, “withdraw”, variants thereof, and the like, may be generally considered with respect to the positioning, direction, and/or operation of various elements relative to a user/operator/manipulator of the device, wherein “proximal” and “withdraw” indicate or refer to closer to or toward the user and “distal” and “advance” indicate or refer to farther from or away from the user. In some instances, the terms “proximal” and “distal” may be arbitrarily assigned in an effort to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device.

The term “extent” may be understood to mean a greatest measurement of a stated or identified dimension, unless the extent or dimension in question is preceded by or identified as a “minimum”, which may be understood to mean a smallest measurement of the stated or identified dimension. For example, “outer extent” may be understood to mean a maximum outer dimension, “radial extent” may be understood to mean a maximum radial dimension, “longitudinal extent” may be understood to mean a maximum longitudinal dimension, etc. Each instance of an “extent” may be different (e.g., axial, longitudinal, lateral, radial, circumferential, etc.) and will be apparent to the skilled person from the context of the individual usage. Generally, an “extent” may be considered a greatest possible dimension measured according to the intended usage, while a “minimum extent” may be considered a smallest possible dimension measured according to the intended usage. In some instances, an “extent” may generally be measured orthogonally within a plane and/or cross-section, but may be, as will be apparent from the particular context, measured differently-such as, but not limited to, angularly, radially, circumferentially (e.g., along an arc), etc. Additionally, the term “substantially” when used in reference to two dimensions being “substantially the same” shall generally refer to a difference of less than or equal to 5%.

The terms “monolithic” and “unitary” shall generally refer to an element or elements made from or consisting of a single structure or base unit/element. A monolithic and/or unitary element shall exclude structure and/or features made by assembling or otherwise joining multiple discrete elements together.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to affect the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously-used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

Pericardial effusion (PE) is a procedural complication risk in left atrial appendage closure (LAAC) especially in a reconstructive approach where device/tissue engagement and reshaping are intrinsic and necessary. In addition, remnant/neo-LAA greater than 10 mm presents a greater future thrombus and embolization risk for the patient. Distally directed force (toward/into the LAA) and strain on the LAA throughout the reshaping and fixation steps involved with inserting a device into the LAA may increase the likelihood of LAA puncture, pericardial effusion, and remnant LAA. In particular, distally directed force used to push an expanded closure device further into the LAA and/or distally directed force applied to a proximal face of the LAA or ostium when engaging a locking mechanism against the ostium of the LAA may pose a risk of PE, LAA rupture, and/or remnant LAA.

The embodiments described below address this concern by providing an implant and system that does not require the operator to push the implant distally and allows the operator to position the device at the ideal deployment position, which reduces systemic tension and strain of the LAA and optimizes implant positioning during the deployment and fixation of the implant to minimize the potential of remnant LAA. The implants described below fix a lock or engagement member at the LAA ostial plane to minimize post implant remnant LAA. The reshaped and occluded LAA is then pulled proximally toward the engagement member fixed at the ostial plane, which reverses the tension and strain applied to the LAA in the reshaping phase of the procedure and completes the deployment at the ostial plane for complete LAA obliteration with no remnant LAA.

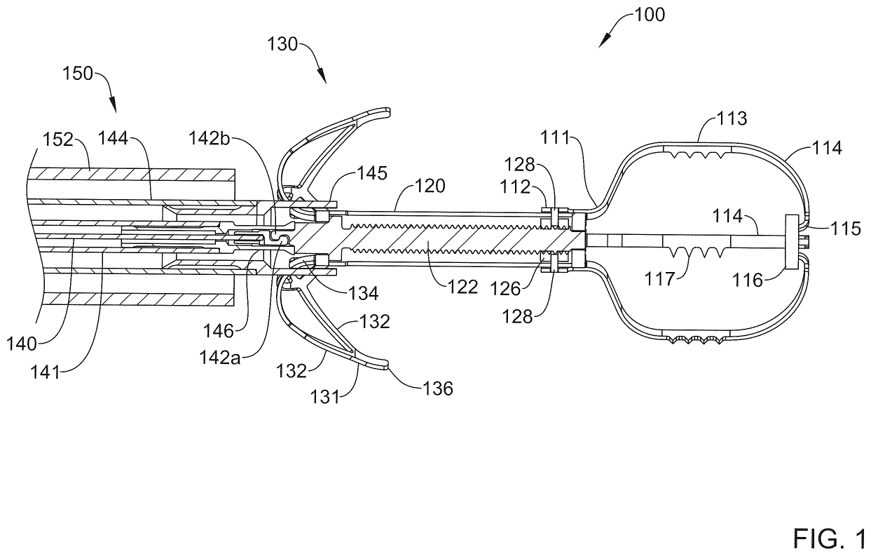

FIG. 1 illustrates an occlusive implant 100 and delivery system 150 for occluding the LAA. The occlusive implant 100 may be reversibly coupled to the delivery system 150 for delivery and actuation. It should be noted that in any given figure, some features of the occlusive implant 100 may not be shown, or may be shown schematically, for simplicity. Additional details regarding some of the components of the occlusive implant 100 may be illustrated in other figures in greater detail. The occlusive implant 100 may include an expandable framework 110, a shaft 120, and a lock 130 coupled to the shaft. The expandable framework 110 may have a proximal end coupled to a collar 112. The collar 112 may be disposed over the distal end of the shaft 120 during delivery and expansion of the expandable framework 110 and lock 130, as shown in FIG. 1. The expandable framework 110 may be configured to expand from a collapsed delivery configuration to an expanded deployed configuration. The expandable framework 110 is shown in the expanded deployed configuration in FIG. 1. The expandable framework may be biased in the expanded deployed configuration, may be self-expandable, and may be made of a shape memory material such as nitinol.

In some embodiments, the expandable framework 110 may include a plurality of struts 114 each having a proximal end 111, a middle portion 113, and distal end 115. The proximal ends 111 may be fixed to the collar 112. In some embodiments, the struts 114 and the collar 112 may be formed as a single monolithic structure. The distal ends 115 of the struts may be coupled to one another or they may be coupled to another structure. In the embodiment shown in FIG. 1, the distal ends 115 of the struts 114 are coupled to a retainer 116. The distal ends 115 may be bent back proximally and coupled to the retainer 116 disposed within an interior space defined by the struts, as shown in FIG. 1. In other embodiments, the distal ends 115 may be coupled together or they may extend distally with the retainer 116 positioned outside the interior space. The middle portions 113 of each strut 114 may be moveable between the collapsed delivery configuration and the expanded deployed configuration, and may be biased in the expanded deployed configuration.

The implant 100 may be delivered through a delivery sheath 152 configured to hold both the expandable framework 110 and the lock 130 in the collapsed delivery configuration. In the collapsed delivery configuration, the expandable framework 110 may be in a substantially cylindrical configuration, with the struts 114 extending substantially linearly from the collar 112. Proximal withdrawal of the delivery sheath 152 or advancement of the implant 100 distally out of the delivery sheath 152 allows the struts 114 to radially expand into the configuration shown in FIG. 1. The expandable framework 110 or at least the struts 114 may be made of a shape memory material, heat set in the expanded configuration.

At least the middle portion 113 of each strut may have a plurality of projections 117 extending laterally from the strut. The projections 117 may be a plurality of teeth 117 as shown in FIG. 1. The teeth 117 may be disposed only on one side of the struts 114 when looking axially down the expandable framework from the shaft 120 to the distal ends 115 of the struts. This configuration allows the struts to engage and grip the inner wall of the LAA when the expandable framework 110 is rotated in a first direction to twist the LAA with the implant, while allowing the expandable framework to slide relative to the inner wall when rotated in a second direction opposite the first direction.

In the embodiments illustrated in FIGS. 1-5, the teeth 117 extend only on the left side of the struts 114 when looking from the proximal ends 111 to the distal ends 115 of the struts, which causes the teeth 117 to grip or become embedded in the inner wall of the LAA when the implant 100 is rotated counter-clockwise. In other embodiments, the projections 117 may extend from both sides of the struts, or radially outward from the outer surface (facing the LAA) of the struts, or any combination thereof.

The shaft 120 may be disposed within the collar 112 and the collar 112 may be axially moveable over the shaft 120. The lock 130 is configured to lock the implant 100 to the LAA and hold the LAA closed. The lock 130 may include an engagement member 131 fixed to the proximal end of the shaft 120. The engagement member 131 may include a plurality of arms 132 where each arm 132 has a first end 134 fixed to the proximal end of the shaft 120 and an opposing second end 136. In the expanded deployed configuration, each arm 132 bends distally with the second end 136 of each arm extending distally and spaced apart radially from the shaft 120, as shown in FIG. 1. In the collapsed delivery configuration each arm 132 extends proximally from the proximal end of the shaft 120. The engagement member 131 may be configured to move from a proximally facing constrained delivery configuration to a distally facing radially expanded configuration, with the engagement member 131 biased in the distally facing radially expanded configuration shown in FIG. 1.

The cross-sectional view in FIG. 1 illustrates the elements of the delivery system, which may include the delivery sheath 152, a rotator sheath 144, an actuation sheath 141, and a core wire 140 removably coupled to the proximal end of a rod 122. The core wire 140 may be configured to rotate the rod 122 and expandable framework 110 together, to move the expandable framework 110 axially relative to the shaft 120. The distal end of the core wire 140 may include a first coupler 142a and the proximal end of the rod 122 may include a second coupler 142b, which together form a two-piece coupling arrangement 142a, 142b. In some embodiments, the coupling arrangement 142a, 142b is a train car latch. A removable cover 146 may be slidingly disposed over at least the two-piece coupling arrangement 142a, 142b and a portion of the core wire 140. The cover 146 may be fixed to the distal end of the actuation sheath 141, and the cover 146 may engage the core wire 140 and the two-piece coupling arrangement 142a, 142b with a friction fit. During delivery, the actuation sheath 141 and cover 146 are in a distally advanced position such that the cover 146 is disposed over the two-piece coupling arrangement 142a, 142b, as shown in FIG. 1, preventing the two-piece coupling arrangement 142a, 142b from becoming disengaged. The actuation sheath 141, core wire 140, and two-piece coupling arrangement 142a, 142b allow for rotation and axial movement of the actuation sheath 141 and core wire 140 to be translated to rotation and axial movement of the occlusive implant 100. The two-piece coupling arrangement 142a, 142b may be configured to be released by lateral movement of the first coupler 142a relative to the second coupler 142b when the cover 146 has been withdrawn proximally off of the first coupler 142a. The covered coupling arrangement allows the core wire 140 to rotate and drive the rod 122 in both a counter-clockwise and a clockwise direction without becoming disengaged from the rod 122.

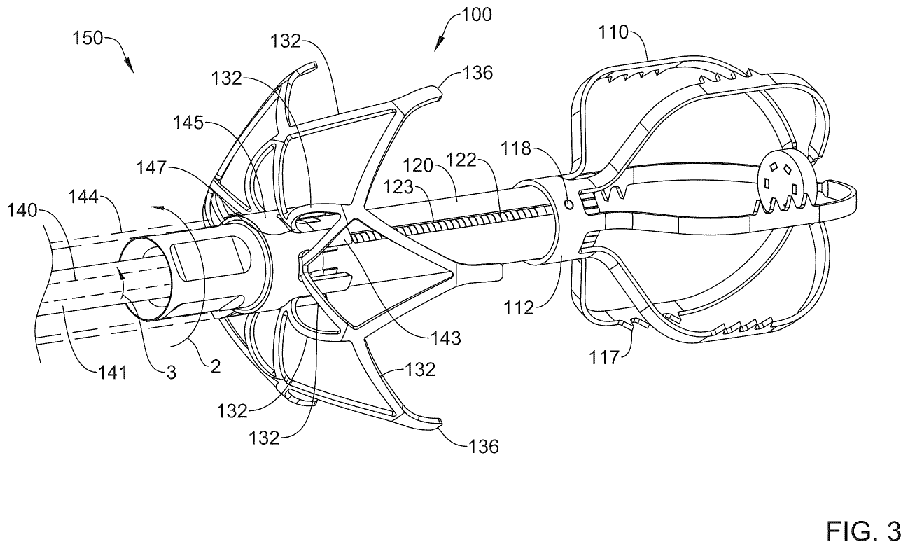

The cross-sectional view in FIG. 1 also illustrates the elements of the shaft 120 which includes the rod 122 and a nut 126 disposed around the rod. The rod 122 may be threaded and threadingly engaged with the nut 126. In other embodiments, the nut 126 may have a smooth inner surface and slide over the threaded rod 122. The nut 126 may have at least one opening in a sidewall thereof, and the collar 112 may have at least one aperture 118 through a sidewall thereof (see FIG. 3), with the opening in the nut 126 and the aperture 118 in the collar 112 aligned such that at least one pin 128 may extend through one aperture in the collar and one opening in the nut. One end of the pin 128 may be disposed within the threading on the rod 122 to secure the collar 112 to the nut 126 and the rod 122 and cause the nut 126, collar 112 and pin 128 to move together along the rod 122 as the rod rotates. In the embodiment shown in FIG. 1, the nut 126 has two openings on opposite sides, the collar 112 has two apertures on opposite sides, and two pins 128 extend through the aligned openings and apertures and engage the threading on the rod 122 to secure the collar 112 to the rod 122. The rod 122 threadingly engages the nut 126 and pins 128 such that rotation of the core wire 140 and actuation sheath 141 in a first direction, such as counter clockwise, causes the expandable framework 110 and collar 112 to be pulled proximally over the shaft 120 with the rod 122 and shaft 120 extending into the interior space defined by the expandable framework 110, and rotation of the core wire 140 and actuation sheath 141 in a second direction, such as clockwise, causes the expandable framework 110 and collar 112 to be pushed distally over the shaft 120. The rotator sheath 144 and shaft 120 remain in place while the core wire 140 and actuation sheath 141 rotate within them.

The rotator sheath 144 extends over the actuation sheath 141 and is axially and rotatably moveable independent of the actuation sheath 141. The distal end of the rotator sheath 144 may include a coupler 145 with a plurality of axially, distally extending fingers 143 (see FIG. 3) with recesses 147 between adjacent fingers, where the fingers 143 are configured to be positioned between the arms 132 of the lock 130 and the recesses 147 engage the proximal ends of the arms 132. With the rotator sheath 144 and coupler 145 thus engaged with the lock 130, rotation of the rotator sheath 144 in the first direction (counter-clockwise in this example), indicated by arrow 2 in FIG. 3, rotates the entire implant 100, including the lock 130, shaft 120, and expandable framework 110 in the first direction, causing the projections 117 on the expandable framework 110 to grip or become embedded in the inner wall of the LAA, twisting the LAA around the implant 100 and closing off the opening of the LAA around the shaft 120.

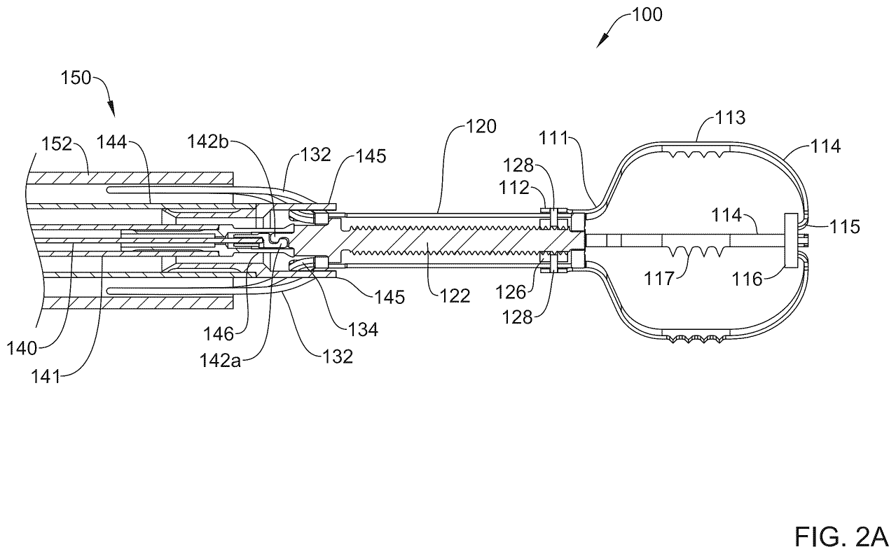

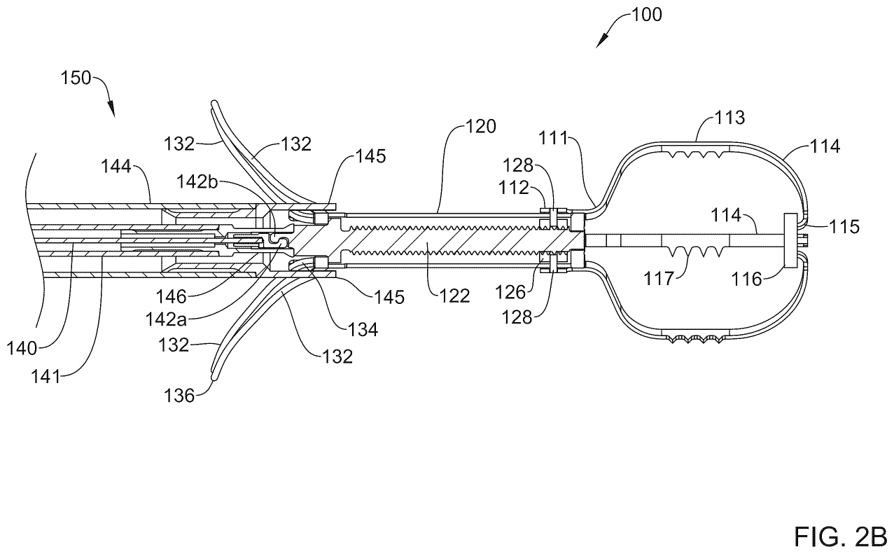

When the expandable framework 110 has been positioned in the desired location within the LAA, the delivery sheath 152 is withdrawn proximally, allowing the expandable framework 110 to fully expand. FIG. 2A shows the implant 100 when the delivery sheath 152 has been partially withdrawn proximally from the arms 132, showing the arms 132 in the proximally facing constrained configuration within the delivery sheath 152. In FIG. 2B, the delivery sheath 152 has been withdrawn fully and the arms 132 have begun to move from their constrained delivery configuration within the delivery sheath 152 to their fully expanded configuration as shown in FIG. 1. As seen in FIG. 2A, in the constrained delivery configuration, the arms 132 extend in the proximal direction, away from the expandable framework 110. As shown in FIG. 2B, the second ends 136 of the arms 132 continue to move distally until they are positioned over the shaft 120 and are pointing in the distally facing orientation shown in FIG. 1. Withdrawal of the delivery sheath 152 proximally from the lock 130 allows the arms 132 to automatically expand and flip over to the distally facing expanded configuration shown in FIG. 1.

In the embodiment shown in the figures, the lock 130 includes an engagement member 131 (see FIG. 1) including a plurality of arms 132, each arm 132 having a first end 134 fixed to the shaft adjacent the proximal end of the shaft 120. In the radially expanded configuration, each arm 132 bends back over the shaft 120 with the second end 136 extending distally from the first end 134 to the second end 136 which faces distally. In some embodiments, the second ends 136 of adjacent arms 132 may be joined to form a point, as shown in FIGS. 1 and 3. The plurality of arms 132 may be interconnected to define a plurality of diamond shapes, as shown in FIG. 3. In some embodiments, each of the plurality of arms 132 may be cut from a tube forming the shaft 120, with the arms 132 heat set in the expanded configuration. In such an embodiment, the shaft 120 and the engagement member 131 are a single monolithic structure.

FIGS. 3-5 show the movement of the expandable framework 110 during implantation. The rotator sheath 144 and core wire 140 are shown in phantom lines. During a method of closing the LAA, the implant 100 is coupled to the delivery system 150 including the delivery sheath 152, rotator sheath 144, actuation sheath 141, and core wire 140. The delivery sheath 152 is not shown, and the rotator sheath 144 is shown transparent in order to see the actuation sheath in FIGS. 3-5. The core wire 140 is coupled to the rod 122 via the two-piece coupling arrangement 142a, 142b as discussed above. When the delivery sheath is withdrawn proximally, the expandable framework 110 automatically expands radially and the engagement member 131 expands and flips distally into the configuration shown in FIG. 3. In the expanded configuration, the collar 112 is positioned over the distal end of shaft 120. The fingers 143 of the coupler 145 may engage the arms 132 of the engagement member 131 such that rotation of the rotator sheath 144 directly rotates the arms 132 and the attached shaft 120 which in turn rotates the expandable framework 110.

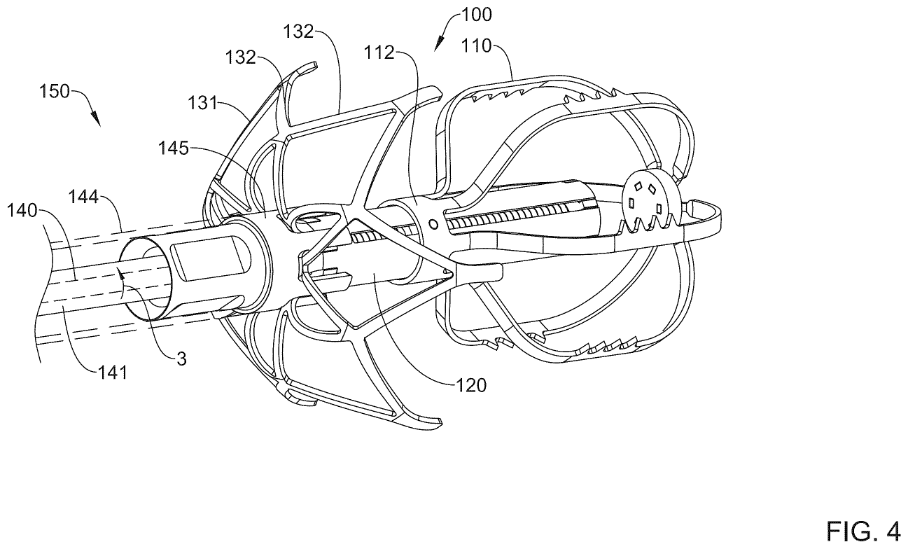

The core wire 140 and actuation sheath 141 may be rotated in the first direction (counter-clockwise in this example), indicated by arrow 3, which causes the rod 122 to rotate, pulling the collar 112 and attached expandable framework 110 proximally, as shown in the difference between FIGS. 3 and 4. In some embodiments, the rotator sheath 144 with its attached coupler 145, and the actuation sheath 141 with its attached cover 146 may be moved axially and rotated independently of one another. The rotator sheath 144 and coupler 145 are configured to rotate the arms 132 which are fixed to the shaft 120 and expandable framework 110 through the collar 112. The collar 112 and attached expandable framework 110 are rotationally fixed to the shaft 120 via the pins 128 which extend through the apertures 118 in the collar and through longitudinal slots 123 in the shaft 120, as shown in FIG. 3. However, the collar 112 and attached expandable framework 110 are axially slidable over the shaft 120 due to the pins 128 extending through the apertures 118 in the collar 112 and the openings in the nut 126. One end of each pin 128 is positioned in the threading on the rod 122. Rotation of the rod 122 causes the pins 128 to travel along the threading on the rod 122, moving the pins, collar 112, and expandable framework 110 axially over the shaft 120. The rotational and axial movements of the expandable framework 110 are thus independently controlled by the rotator sheath and actuation sheath, respectively.

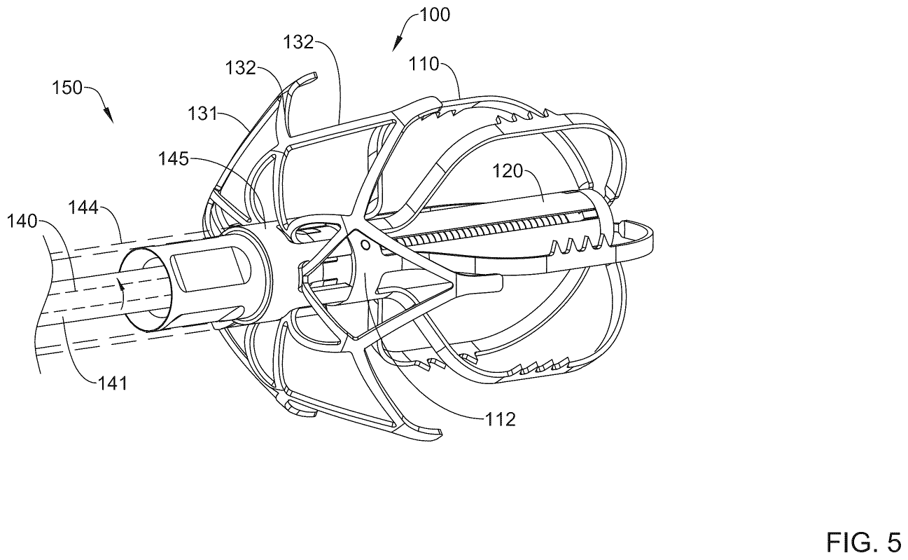

Continued rotation of the actuation sheath 141 pulls the collar 112 and expandable framework 110 further proximally over the shaft 120 and rod 122, as shown in FIGS. 4 and 5. As the expandable framework 110 is pulled proximally, the shaft 120 extends through the void within the expandable framework, as shown in FIG. 5, and the proximal edge of the expandable framework 110 resides adjacent the engagement member 131. The twisted neck of the LAA may be pinched and compressed between the expandable framework 110 and the engagement member 131, as described below.

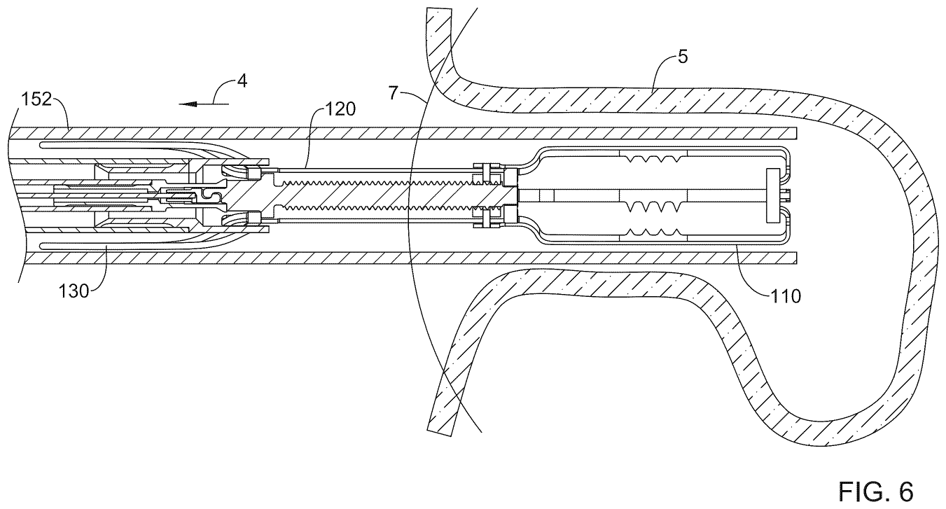

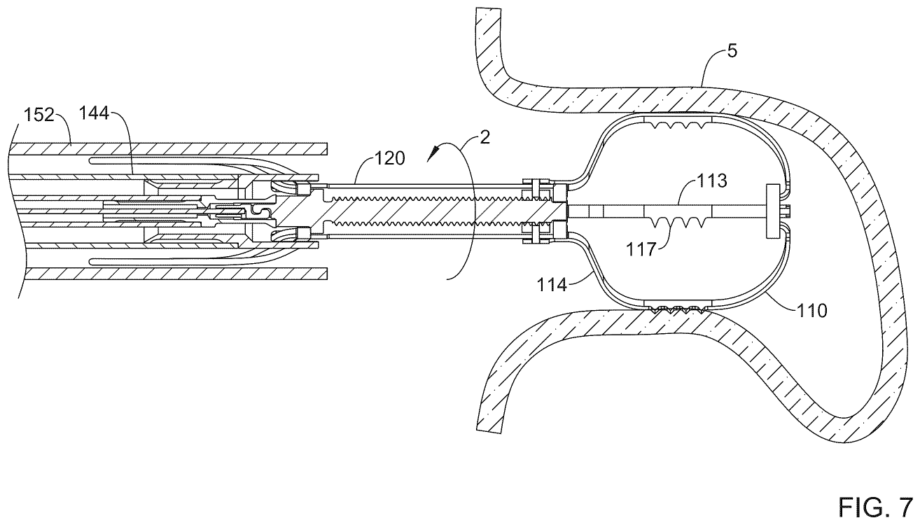

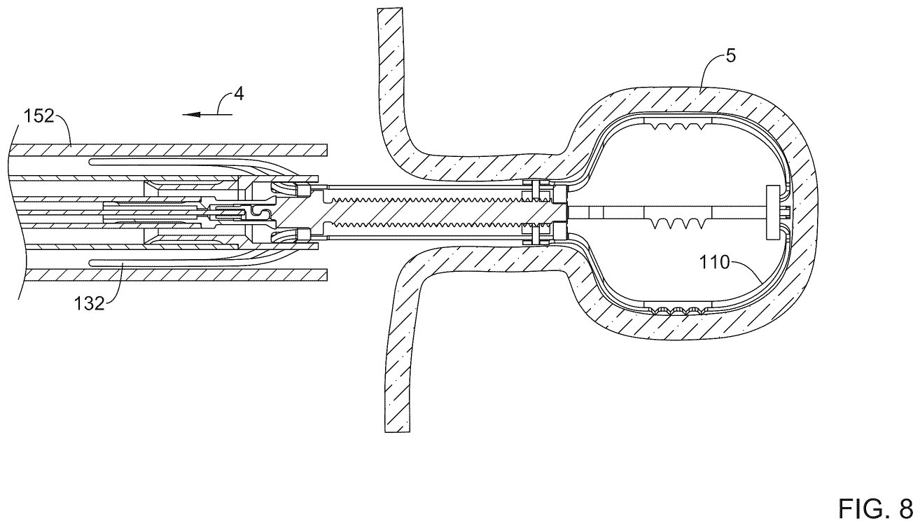

FIGS. 6-12 illustrate the steps in a method for closing the LAA 5 with the implant 100. The implant 100 is coupled to the delivery system 150, with the expandable framework 110 and lock 130 both in collapsed or constrained delivery configuration inside the delivery sheath 152. In the first step, shown in FIG. 6, the delivery sheath 152 with the implant disposed therein is delivered through the patient's vasculature until only the expandable framework 110 is positioned inside the LAA 5 while the lock 130 remains proximal of the LAA, and the shaft 120 spans the ostial plane 7. The delivery sheath 152 is withdrawn proximally, indicated by arrow 4, until the distal end of the delivery sheath 152 is proximal of the expandable framework 110, which automatically expands against the walls of the LAA 5, as shown in FIG. 7. The delivery sheath 152 remains over the lock 130, thus the expandable framework 110 is expandable independently of the lock 130. The expandable framework 110 may be positioned such that at least the middle portion 113 of each strut 114 is in contact with the inner walls of the LAA 5. The rotator sheath 144 and attached coupler 145 is rotated in a first direction (counter-clockwise in this example), indicated by arrow 2, causing the projections 117 on the struts 114 to grip and/or become embedded within the wall of the LAA 5. The expandable framework 110 may be rotated 180 degrees to 360 degrees to twist the LAA 5 around the shaft 120 and close off the opening or neck of the LAA 5 around the shaft 120, as shown in FIG. 8.

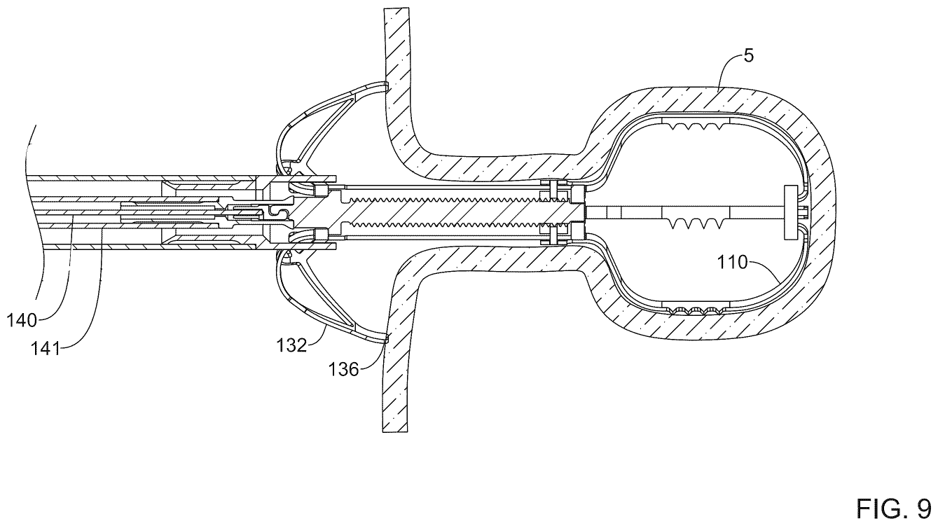

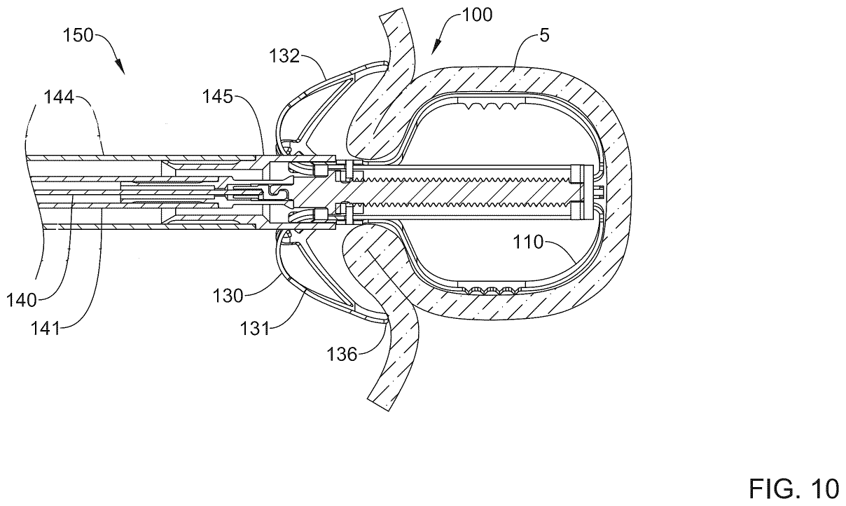

With the LAA 5 compressed and twisted around the shaft 120, the delivery sheath 152 is withdrawn proximally from the lock 130, indicated by arrow 4, allowing the arms 132 of the engagement member 131 to flip from their constrained proximally extending position (FIG. 8) to their radially expanded distally extending position with the second ends 136 of the arms 132 engaging the proximal face of the LAA 5, as shown in FIG. 9. To actuate the lock 130, the actuation sheath 141 and core wire 140 are rotated which pulls the expandable framework 110 toward the lock 130, as discussed above. The expandable framework 110 moves axially independently of the lock 130. FIG. 10 shows the twisted neck of the LAA 5 pinched or compressed between the expandable framework 110 and the lock 130. This proximal movement of the expandable framework 110 within the LAA 5 may avoid a remnant pocket. In some embodiments, the rod 122 has 80 threads per inch, with 33 revolutions of the actuation sheath 141 and core wire 140 resulting in proximal movement of the expandable framework 110 0.41 inches (1.04 centimeters) toward the lock 130 for 100% actuation. The threading may be a single start thread. In other embodiments, the thread may have 2-3 starts. In this fully locked configuration, the collar 112 has moved proximally over the shaft 120 such that the shaft is disposed within the interior space defined by the expandable framework 110, and the collar 112 is disposed over the proximal end of the shaft 120. The second ends 136 of the arms may become embedded in the twisted tissue at the proximal face of the LAA 5. The second ends 136 of the arms 132 may prevent the tissue from untwisting, thereby holding the implant within the LAA 5 and maintain the twist of tissue that completely closes off the LAA 5.

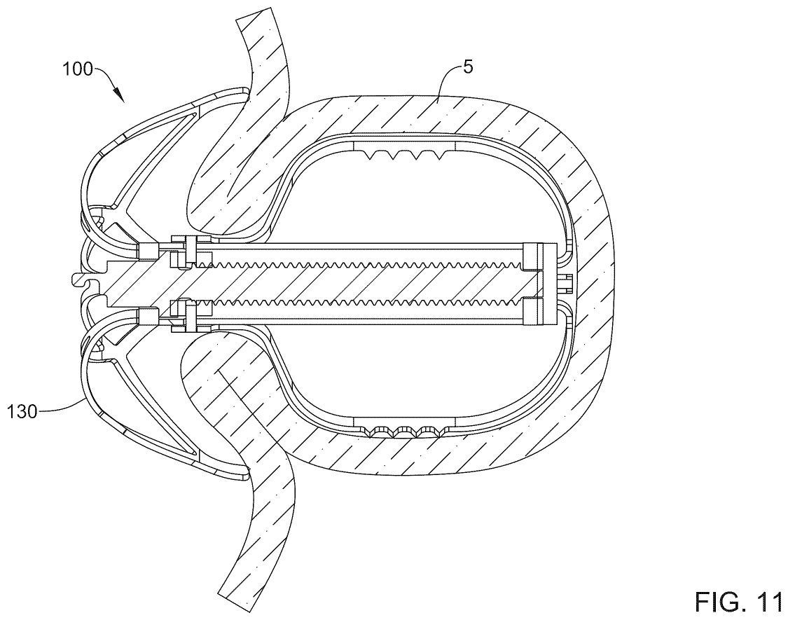

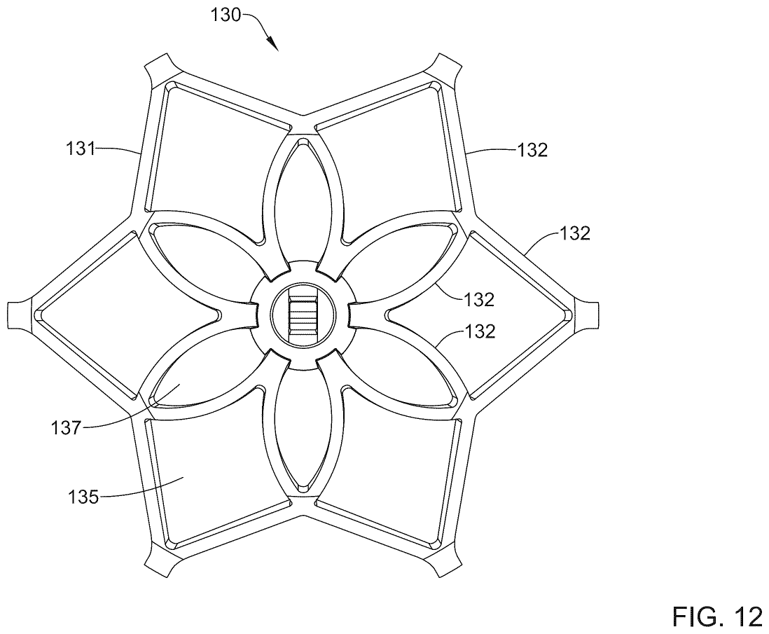

Once the implant 100 is in the fully locked configuration, as shown in FIG. 10, the entire delivery system 150, including the delivery sheath 152, the rotator sheath 144 and coupler 145, and the actuation sheath 141 and attached cover 146 may be withdrawn proximally relative to the core wire 140, uncovering the two-piece coupling arrangement 142a, 142b. The core wire 140 with its first coupler 142a may be moved laterally relative to the second coupler 142b, thereby uncoupling the first coupler 142a from the second coupler 142b, and allowing the core wire 140 to be withdrawn proximally, leaving the implant 100 inserted in the closed LAA 5, as shown in FIG. 11. FIG. 12 shows a proximal end view of the implanted implant of FIG. 11, showing the expanded lock 130 engaged over the twisted proximal face of the LAA. The arms 132 of the engagement member 131 form a star shape made up of a plurality of interconnected diamond and/or petal shapes. In the embodiment shown in FIG. 12, the plurality of arms 132 form a series of inner openings 137 and a series of outer openings 135. As shown, six inner openings 137 are offset from six outer openings 135.

Optimal reconstructive implant fixation and seal for LAAC may vary depending on the individual left atrium complex such as the limbus ridge, mitral annulus, and LAA size and morphology. In addition to the interconnected lock 130 described above, a fully or partially individually actuatable lock may conform to more complex geometry at the LAA opening. In some instances, structures adjacent to the LAA opening may interfere with complete engagement of the lock with the tissue. Such adjacent structure may include the limbus, vestibule, and ridge between the LAA and left superior pulmonary vein (LSPV) and left inferior pulmonary vein (LIPV). The fixation force of the lock 130 may be directly proportional to the size and thickness of the arms 132, however increasing the size and/or thickness of the arms may interfere with the left atrium function.

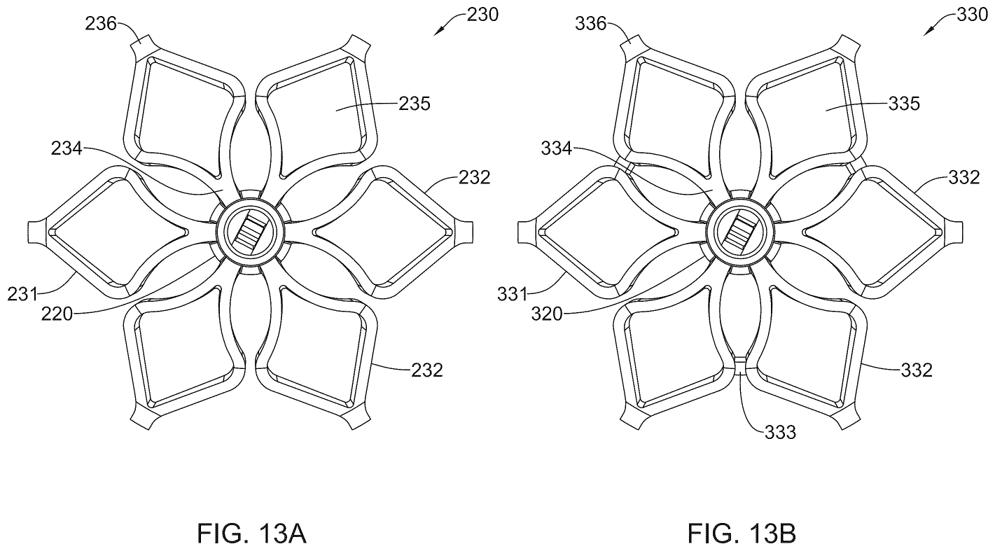

FIGS. 13A and 13B illustrate alternative locks 230, 330 in the radially expanded configuration engaged over the twisted proximal face of the LAA. The locks 230, 330 involve independent and non-planar fixation and compression elements that achieve an increased actuation range and higher compression based on increased device to tissue surface contact which may secure the tissue twist and promote endothelization. In each embodiment, the lock 230, 330 differs from lock 130 described above, but all other elements of the implant are as described above.

In each of FIGS. 13A and 13B, the lock 230, 330 includes an engagement member 231, 331 including a plurality of arms 232, 332 forming a plurality of petals each having a base 234, 334 fixed to the proximal end of the shaft 220, 320 and an opposing free end 236, 336. Each petal may be defined by at least two arms 232, 332 extending between the base 234, 334 and the free end 236, 336 with an opening therebetween. The arms 232, 332 forming each petal may be connected only at the base 234, 334 and free end 236, 336 to define a single opening between the arms. In other embodiments, one or more connecting strut (not shown) may extend between the arms 232, 332 of a single petal to form a plurality of openings within the single petal. The arms 232, 332 forming each petal extend radially away from the shaft 220, 320 and are spaced apart from one another along their middle regions to define the opening therebetween in the radially expanded configuration, as shown in FIGS. 13A and 13B. The arms 232, 332 may form petal shapes disposed circumferentially around the shaft 220, 320.

In the embodiment shown in FIG. 13A, each petal may be independently bendable at base 234 in an axial direction relative to the shaft 220. Each arm 232 is only connected to one adjacent arm 232 at the base 234 and free end 236 to form a petal. Thus, each petal is devoid of any connection with adjacent petals such that the petals are all independently movable relative to the shaft 220 and each other. Each petal may bend relative to the shaft 220 where the base 234 joins the shaft 220 to a different degree compared to the other petals. This independent movement may allow the lock 230 to conform to an uneven surface at the twisted proximal LAA face.

FIG. 13B shows a lock 330 that is the same as lock 230 but with added couplers 333 joining adjacent arms 223 of circumferentially adjacent petals. The couplers 333 may be disposed at a middle region of the arms 332 and create a lock structure in which two adjacent petals move together relative to the shaft 320. In this manner, three sets of two petals move independently of one another in order to conform to the twisted tissue at the proximal face of the LAA.

In any of the locks 130, 230, 330 described above, the width and thickness of the arms 132, 232, 332 may be varied to reduce strain and achieve desired loading and deploying forces. In any of the above described embodiments, all of the struts 132, 232, 332 forming the lock 130, 230, 330 may have the same thickness and width, or individual struts may have a different with and/or thickness.

The materials that can be used for the various components of the system (and/or other elements disclosed herein) and the various components thereof disclosed herein may include those commonly associated with medical devices and/or systems. For simplicity purposes, the following discussion refers to the system. However, this is not intended to limit the devices and methods described herein, as the discussion may be applied to other elements, members, components, or devices disclosed herein, such as, but not limited to, the occlusive implant, the delivery sheath, the core wire, the expandable framework, the rod, the elongate fingers, the couplers, the lock, the projections, etc. and/or elements or components thereof.

In some embodiments, the system and/or components thereof may be made from a metal, metal alloy, polymer, a metal-polymer composite, ceramics, combinations thereof, and the like, or other suitable material. Some examples of suitable polymers may include polytetrafluoroethylene (PTFE), ethylene tetrafluoroethylene (ETFE), fluorinated ethylene propylene (FEP), polyoxymethylene (POM; for example, DELRIN®), polyether block ester, polyurethane, polypropylene (PP), polyvinylchloride (PVC), polyether-ester (for example, ARNITEL®), ether or ester based copolymers (for example, butylene/poly (alkylene ether) phthalate and/or other polyester elastomers such as HYTREL®), polyamide (for example, DURETHAN® or CRISTAMID®), elastomeric polyamides, block polyamide/ethers, polyether block amide (PEBA; for example, PEBAX®), ethylene vinyl acetate copolymers (EVA), silicones, polyethylene (PE), MARLEX® high-density polyethylene, MARLEX® low-density polyethylene, linear low density polyethylene (for example, REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID®), perfluoro (propyl vinyl ether) (PFA), ethylene vinyl alcohol, polyolefin, polystyrene, epoxy, polyvinylidene chloride (PVdC), poly (styrene-b-isobutylene-b-styrene) (for example, SIBS and/or SIBS 50A), polycarbonates, polyurethane silicone copolymers (for example, Elast-Eon® or ChronoSil®), biocompatible polymers, other suitable materials, or mixtures, combinations, copolymers thereof, polymer/metal composites, and the like. In some embodiments, the system and/or components thereof can be blended with a liquid crystal polymer (LCP). For example, the mixture can contain up to about 6 percent LCP.

Some examples of suitable metals and metal alloys include stainless steel, such as 304 and/or 316 stainless steel and/or variations thereof, mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; platinum; palladium; gold; combinations thereof, or any other suitable material.

In at least some embodiments, portions or all of the system and/or components thereof may also be doped with, made of, or otherwise include a radiopaque material. Radiopaque materials are understood to be materials capable of producing a relatively bright image on a fluoroscopy screen or another imaging technique (e.g., ultrasound, etc.) during a medical procedure. This relatively bright image aids the user of the system in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the system to achieve the same result.

In some embodiments, the system and/or components thereof may include a fabric material. The fabric material may be composed of a biocompatible material, such a polymeric material or biomaterial, adapted to promote tissue ingrowth. In some embodiments, the fabric material may include a bioabsorbable material. Some examples of suitable fabric materials include, but are not limited to, polyethylene glycol (PEG), nylon, polytetrafluoroethylene (PTFE, ePTFE), a polyolefinic material such as a polyethylene, a polypropylene, polyester, polyurethane, and/or blends or combinations thereof.

In some embodiments, the system and/or components thereof may include and/or be formed from a textile material. Some examples of suitable textile materials may include synthetic yarns that may be flat, shaped, twisted, textured, pre-shrunk or un-shrunk. Synthetic biocompatible yarns suitable for use in the present disclosure include, but are not limited to, polyesters, including polyethylene terephthalate (PET) polyesters, polypropylenes, polyethylenes, polyurethanes, polyolefins, polyvinyls, polymethylacetates, polyamides, naphthalene dicarboxylene derivatives, natural silk, and polytetrafluoroethylenes. Moreover, at least one of the synthetic yarns may be a metallic yarn or a glass or ceramic yarn or fiber. Useful metallic yarns include those yarns made from or containing stainless steel, platinum, gold, titanium, tantalum or a Ni—Co—Cr-based alloy. The yarns may further include carbon, glass or ceramic fibers. Desirably, the yarns are made from thermoplastic materials including, but not limited to, polyesters, polypropylenes, polyethylenes, polyurethanes, polynaphthalenes, polytetrafluoroethylenes, and the like. The yarns may be of the multifilament, monofilament, or spun types. The type and denier of the yarn chosen may be selected in a manner which forms a biocompatible and implantable prosthesis and, more particularly, a vascular structure having desirable properties.

In some embodiments, the system and/or components thereof may include and/or be treated with a suitable therapeutic agent. Some examples of suitable therapeutic agents may include anti-thrombogenic agents (such as heparin, heparin derivatives, urokinase, and PPack (dextrophenylalanine proline arginine chloromethyl ketone)); anti-proliferative agents (such as enoxaparin, angiopeptin, monoclonal antibodies capable of blocking smooth muscle cell proliferation, hirudin, and acetylsalicylic acid); anti-inflammatory agents (such as dexamethasone, prednisolone, corticosterone, budesonide, estrogen, sulfasalazine, and mesalamine); antineoplastic/antiproliferative/anti-mitotic agents (such as paclitaxel, 5-fluorouracil, cisplatin, vinblastine, vincristine, epothilones, endostatin, angiostatin and thymidine kinase inhibitors); anesthetic agents (such as lidocaine, bupivacaine, and ropivacaine); anti-coagulants (such as D-Phe-Pro-Arg chloromethyl ketone, an RGD peptide-containing compound, heparin, anti-thrombin compounds, platelet receptor antagonists, anti-thrombin antibodies, anti-platelet receptor antibodies, aspirin, prostaglandin inhibitors, platelet inhibitors, and tick antiplatelet peptides); vascular cell growth promoters (such as growth factor inhibitors, growth factor receptor antagonists, transcriptional activators, and translational promoters); vascular cell growth inhibitors (such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin); immunosuppressants (such as the “olimus” family of drugs, rapamycin analogues, macrolide antibiotics, biolimus, everolimus, zotarolimus, temsirolimus, picrolimus, novolimus, myolimus, tacrolimus, sirolimus, pimecrolimus, etc.); cholesterol-lowering agents; vasodilating agents; and agents which interfere with endogenous vasoactive mechanisms.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The disclosure's scope is, of course, defined in the language in which the appended claims are expressed.

What is claimed is:

1. An implant for left atrial appendage closure, comprising:
an expandable framework having a proximal end coupled to a collar, the expandable framework configured to expand from a collapsed delivery configuration to an expanded deployed configuration;
a shaft disposed within the collar wherein the collar is axially moveable relative to the shaft;
a rod disposed within the shaft and coupled to the collar; and
a lock coupled to the shaft, the lock including an engagement member configured to move between a proximally facing constrained configuration and a distally facing radially expanded configuration;
wherein the rod is coupled to the collar such that rotation of the rod pulls the collar and expandable framework proximally over the shaft toward the lock, wherein the expandable framework is configured to rotate and move axially independently of the lock.

2. The implant of claim 1, wherein the rod is threaded and the implant further comprises a nut threadingly engaged around the rod and coupled to the collar.

3. The implant of claim 2, wherein the collar and nut each have at least one opening and the openings are aligned, the implant further including at least one pin extending through the openings in the collar and nut and having an end positioned in the threading on the rod.

4. The implant of claim 1, wherein the engagement member is biased in the distally facing radially expanded configuration.

5. The implant of claim 1, wherein the engagement member is fixed to the proximal end of the shaft and includes a plurality of arms.

6. The implant of claim 5, wherein each arm has a first end fixed to the proximal end of the shaft and an opposing second end, wherein in the collapsed delivery configuration each arm extends proximally from the proximal end of the shaft, and in the expanded deployed configuration, each arm bends distally with the second end of each arm extending distally and spaced apart radially from the shaft.

7. The implant of claim 6, wherein the plurality of arms is interconnected to define a plurality of diamond shapes.

8. The implant of claim 5, wherein the plurality of arms forms a plurality of petals each having a base fixed to the shaft and an opposing free end, each petal defined by two arms extending between the base and the free end with an opening therebetween.

9. The implant of claim 8, wherein each petal is independently movable relative to the shaft and adjacent petals, each petal being free of connection to any adjacent petal.

10. The implant of claim 1, wherein after the expandable framework is moved to the expanded deployed configuration, the expandable framework is configured to be rotated and then pulled proximally over the shaft.

11. The implant of claim 1, wherein the expandable framework includes a plurality of struts each having a proximal end, a middle portion, and distal end, wherein the proximal ends are coupled to the collar, the distal ends are coupled together, and the middle portions are moveable between the collapsed delivery configuration and the expanded deployed configuration, wherein the plurality of struts are biased in the expanded deployed configuration.

12. The implant of claim 11, wherein at least the middle portion of each strut has a plurality of projections extending laterally from the strut.

13. An implant assembly for left atrial appendage closure, comprising:
a delivery sheath defining a lumen;
a rotator sheath slidably disposed within the lumen of the delivery sheath;
an actuation sheath disposed within a lumen of the rotator sheath;
an implant releasably coupled to a distal end of the rotator sheath and the actuation sheath, the implant including:
an expandable framework configured to expand from a collapsed delivery configuration to an expanded deployed configuration;
a shaft slidably disposed within at least a portion of the expandable framework;
a rod disposed within the shaft and coupled to the expandable framework and the actuation sheath; and
a lock coupled to a proximal end of the shaft, the lock including an engagement member configured to move between a proximally facing constrained configuration and a distally facing radially expanded configuration.

14. The implant assembly of claim 13, wherein the distal end of the rotator sheath has a plurality of distally extending fingers configured to engage the engagement member.

15. The implant assembly of claim 13, wherein the actuation sheath is configured to be rotated to rotate the rod which pulls the expandable framework proximally over the shaft and rod.

16. The implant assembly of claim 15, wherein the rod is threaded and has a proximal end releasably coupled to the actuation sheath.

17. The implant assembly of claim 13, wherein the engagement member is fixed to the proximal end of the shaft and includes a plurality of arms.

18. The implant assembly of claim 17, wherein each arm has a first end fixed to the proximal end of the shaft and an opposing second end, wherein in the collapsed delivery configuration each arm extends proximally from the proximal end of the shaft, and in the expanded deployed configuration, each arm bends distally with the second end of each arm extending distally and spaced apart radially from the shaft.

19. The implant assembly of claim 18, wherein the plurality of arms is interconnected to define a plurality of diamonds forming a star shape.

20. A method of closing a left atrial appendage, comprising:
coupling a delivery system to an implant, the implant including:
an expandable framework having a proximal end coupled to a collar, the expandable framework configured to expand from a collapsed delivery configuration to an expanded deployed configuration, at least a portion of the expandable framework having a plurality of projections extending laterally therefrom;
a shaft disposed within the collar and axially moveable relative to the collar;
a lock coupled to the shaft, the lock including an engagement member configured to move between a proximally facing constrained configuration and a distally facing radially expanded configuration;
wherein the expandable framework is in the collapsed delivery configuration and the engagement member is in the proximally facing constrained configuration within the delivery system;
inserting the delivery system through a patient's vasculature until the expandable framework, in the collapsed delivery configuration, is positioned inside the left atrial appendage;
withdrawing the delivery system from the expandable framework to allow the expandable framework to expand to the expanded deployed configuration;
rotating at least a portion of the delivery system to rotate the expandable framework and engage the plurality of projections with an inner surface of the left atrial appendage and twist a neck of the left atrial appendage around the shaft;
withdrawing the delivery system from the lock to allow the engagement member to move into the distally facing radially expanded configuration adjacent a proximal face of the twisted neck of the left atrial appendage;
pulling the expandable framework proximally until the twisted neck of the left atrial appendage is pinched between the expandable framework and the engagement member; and
disengaging the delivery system from the implant.
